SEQUENCE LISTING (1 sequence)

SEQ ID NO: 1 - DNA, 20 bp, synthetic construct
  source 1..20: mol_type = other DNA
  modified_base 1: mod_base = OTHER; note = 2'-O-methoxyethyl ribonucleoside with phosphorothioate linkage
  modified_base 2: mod_base = OTHER; note = 2'-O-methoxyethyl ribonucleoside with phosphorothioate linkage
  modified_base 3: mod_base = OTHER; note = 2'-O-methoxyethyl ribonucleoside with phosphorothioate linkage
  modified_base 4: mod_base = OTHER; note = 2'-O-methoxyethyl ribonucleoside with phosphorothioate linkage
  modified_base 5: mod_base = OTHER; note = 2'-O-methoxyethyl ribonucleoside with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = 2'-O-methoxyethyl ribonucleoside with phosphorothioate linkage
  modified_base 17: mod_base = OTHER; note = 2'-O-methoxyethyl ribonucleoside with phosphorothioate linkage
  modified_base 18: mod_base = OTHER; note = 2'-O-methoxyethyl ribonucleoside with phosphorothioate linkage
  modified_base 19: mod_base = OTHER; note = 2'-O-methoxyethyl ribonucleoside with phosphorothioate linkage
  modified_base 20: mod_base = OTHER; note = 2'-O-methoxyethyl ribonucleoside
  misc_feature 1..5: note = RNA
  misc_feature 6..15: note = DNA
  misc_feature 16..20: note = RNA
atcagtcttttcctctacga

(19) United States
(12) Patent Application Publication
(10) Pub. No.: US 2025/0101417 A1
(43) Pub. Date: Mar. 27, 2025

(54) Implantable Medical Device for the Delivery of a Nucleic Acid

(71) Applicant: Celanese EVA Performance Polymers LLC, Irving, TX (US)

(72) Inventors: Jeffrey C. Haley, Norwood, OH (US); Vijay Gyanani, Powell, OH (US); Brian D. Wilson, Deerfield, IL (US); Harsh Patel, Union, KY (US); Narsi Devanathan, Union, KY (US)

(21) Appl. No.: 18/474,292

(22) Filed: Sep. 26, 2023

(51) Int. Cl. C12N 15/113; A61K 9/127

(52) U.S. Cl. CPC C12N 15/113; A61K 9/1273; C12N 2310/11

(57) ABSTRACT

An implantable medical device is provided. The implantable medical device includes a core having an antisense oligonucleotide (ASO) dispersed within a core polymer matrix. The core polymer matrix includes an ethylene vinyl acetate copolymer. The ASO includes one or more nucleosides attached via internucleoside linkages. At least 10% of the internucleoside linkages comprise chemically modified internucleoside linkages. The implantable device is capable of releasing about 5% to about 60% of the ASO after a time period of about seven days.

### RELATED APPLICATION

The present application is based upon and claims priority to U.S. Provisional Patent Application Ser. No. 63/411,166, having a filing date of Sep. 29, 2022, which is incorporated herein by reference.

### BACKGROUND OF THE DISCLOSURE

Nucleic acids, such as mRNA and siRNA, have recently become a focal point for a substantial amount of gene therapy treatments, such as oncological treatments, vaccines, and so forth. For example, compared to DNA, ribonucleic acids (e.g., mRNA) are not stably integrated into the genome of the transfected cell, thus eliminating the concern that the introduced genetic material will disrupt the normal functioning of an essential gene. Extraneous promoter sequences are also not required for effective translation of the encoded protein, again avoiding possible deleterious side effects. One problem with ribonucleic acid-based gene therapy, however, is that it is far less stable than DNA, especially when it reaches the cytoplasm of a cell and is exposed to degrading enzymes. The presence of a hydroxyl group on the second carbon of the sugar moiety in mRNA, for example, causes steric hindrance that prevents the mRNA from forming the more stable double helix structure of DNA, and thus makes the mRNA more prone to hydrolytic degradation. In light of the above, ribonucleic acids are generally encapsulated into lipid particles (e.g., liposomes, solid lipid particles, etc.) to protect them from extracellular RNase degradation and simultaneously promote cellular uptake and endosomal escape. Unfortunately, however, problems nevertheless remain for their use in many applications. For example, it is difficult to controllably deliver nucleic acid-encapsulated lipid particles over a sustained period of time. One of the reasons for this difficulty is that the lipids employed in the particles tend to have a relatively low melting point, making it difficult to incorporate them into the processes and polymer materials used to form most conventional implantable medical devices.

As such, a need continues to exist for an implantable delivery device that is capable of delivering a nucleic acid over a sustained period of time.

### SUMMARY OF THE DISCLOSURE

In accordance with one embodiment of the present disclosure, an implantable medical device is disclosed.

An implantable medical device is provided. The implantable medical device includes a core having an antisense oligonucleotide (ASO) dispersed within a core polymer matrix. The core polymer matrix includes an ethylene vinyl acetate copolymer. The ASO includes one or more nucleosides attached via internucleoside linkages. At least 10% of the internucleoside linkages comprise chemically modified internucleoside linkages. The implantable device is capable of releasing about 5% to about 60% of the ASO after a time period of about seven days.

Other features and aspects of the present disclosure are set forth in greater detail below.

### BRIEF DESCRIPTION OF THE DRAWINGS

A full and enabling disclosure of the present disclosure, including the best mode thereof, directed to one of ordinary skill in the art, is set forth more particularly in the remainder of the specification, which makes reference to the appended drawings in which:

FIG. 1 is a perspective view of one embodiment of the implantable medical device of the present disclosure;

FIG. 2 is a cross-sectional view of the implantable medical device of FIG. 1;

FIG. 3 is a perspective view of another embodiment of the implantable medical device of the present disclosure;

FIG. 4 is a cross-sectional view of the implantable medical device of FIG. 3;

FIG. 5 is a graph showing the percent release of an antisense oligonucleotide (ASO) for Example 1;

FIG. 6 is a graph showing the surface area normalized release of the ASO for Example 1;

FIG. 7 illustrates HPLC chromatograms taken at various time points for Example 1;

FIG. 8 is a graph showing the percent release of an ASO for Examples 2-9; and

FIG. 9 is a graph showing the surface area normalized release of the ASO for Examples 2-9.

Repeat use of references characters in the present specification and drawing is intended to represent same or analogous features or elements of the disclosure.

### DETAILED DESCRIPTION

It is to be understood by one of ordinary skill in the art that the present discussion is a description of exemplary embodiments only and is not intended as limiting the broader aspects of the present disclosure.

Generally speaking, the present disclosure is directed to an implantable medical device that is capable of delivering a nucleic acid (e.g., an ASO) to a patient (e.g., human, pet, farm animal, racehorse, etc.) over a sustained period of time to help prohibit and/or treat a condition, disease, and/or cosmetic state of the patient. The implantable medical device includes an ASO dispersed within a polymer matrix, which includes one or more ethylene vinyl acetate copolymers. The ASO includes one or more nucleosides attached via internucleoside linkages and at least 10% of the internucleoside linkages comprise chemically modified internucleoside linkages. Further, at a time period of about seven days from about 5% to about 60% of the ASO is released from the implantable medical device.

The ethylene vinyl acetate copolymer(s) employed within the polymer matrix are selected to have a certain melting temperature and melt flow index to help minimize the risk of nucleic acid degradation during processing. The ethylene vinyl acetate copolymer(s) and resulting polymer matrix may, for instance, have a melting temperature of from about 20° C. to about 100° C., in some embodiments from about 25° C. to about 80° C., in some embodiments from about 30° C. to about 70° C., in some embodiments from about 35° C. to about 65° C., and in some embodiments, from about 40° C. to about 60° C., such as determined in accordance with ASTM D3418-15. The melt flow index of the ethylene vinyl acetate copolymer(s) and the resulting polymer matrix may also be from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms.

Various embodiments of the present disclosure will now be described in more detail.

### I. Core Polymer Matrix

The device includes a core having a core polymer matrix including a polymer that is generally hydrophobic in nature so that it can retain its structural integrity for a certain period of time when placed in an aqueous environment, such as the body of a mammal, and stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also suitable for use in the core polymer matrix. Typically, the melt flow index of the hydrophobic polymer ranges from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-13 at a temperature of 190° C. and a load of 2.16 kilograms.

In certain embodiments, the core polymer matrix may contain a semi-crystalline olefin copolymer. The melting temperature of such an olefin copolymer may, for instance, range from about 40° C. to about 140° C., in some embodiments from about 50° C. to about 125° C., and in some embodiments, from about 60° C. to about 120° C., as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, certain aspects of the copolymer can be selectively controlled to help achieve the desired release properties. For instance, the polar monomeric content of the copolymer may be selectively controlled to be within a range of from about 10 wt. % to about 60 wt. %, in some embodiments about 20 wt. % to about 60 wt. %, and in some embodiments, from about 25 wt. % to about 50 wt. %. Conversely, the olefin monomeric content of the copolymer may likewise be within a range of from about 40 wt. % to about 90 wt. %, in some embodiments about 40 wt. % to about 80 wt. %, and in some embodiments, from about 50 wt. % to about 75 wt. %.

In one particular embodiment, for example, the core polymer matrix may contain at least one ethylene vinyl acetate polymer, which is a copolymer that is derived from at least one ethylene monomer and at least one vinyl acetate monomer. In certain cases, the present inventors have discovered that certain aspects of the copolymer can be selectively controlled to help achieve the desired release properties. For instance, the vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. In certain embodiments, the vinyl acetate content ranges from about 25 wt. % to about 32 wt. %. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments from about 1 to about 200 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA® 4030AC); Dow under the designation ELVAX® (e.g., ELVAX® 40W); and Arkema under the designation EVATANE® (e.g., EVATANE 40-55). In embodiments, the ethylene vinyl acetate copolymer in the core polymer matrix is from about 20 wt. % to about 90 wt. %, such as from about 30 wt. % to about 80 wt. %, such as from about 40 wt. % to about 70 wt. %.

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al.; U.S. Pat. No. 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain embodiments, it may also be desirable to employ blends of an ethylene vinyl acetate copolymer and another hydrophobic polymer such that the overall blend and polymer matrix have a melting temperature and/or melt flow index within the range noted above. For example, the polymer matrix may contain a first ethylene vinyl acetate copolymer and a second ethylene vinyl acetate copolymer having a melting temperature that is greater than the melting temperature of the first copolymer. The second copolymer may likewise have a melt flow index that is the same, lower, or higher than the corresponding melt flow index of the first copolymer. The first copolymer may, for instance, have a melting temperature of from about 20° C. to about 60° C., in some embodiments from about 25° C. to about 55° C., and in some embodiments, from about 30° C. to about 50° C., such as determined in accordance with ASTM D3418-15, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190° C. and a load of 2.16 kilograms. The second copolymer may likewise have a melting temperature of from about 50° C. to about 100° C., in some embodiments from about 55° C. to about 90° C., and in some embodiments, from about 60° C. to about 80° C., such as determined in accordance with ASTM D3418-15, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190° C. and a load of 2.16 kilograms. The first copolymer may constitute from about 20 wt. % to about 80 wt. %, in some embodiments from about 30 wt. % to about 70 wt. %, and in some embodiments, from about 40 wt. % to about 60 wt. % of the polymer matrix, and the second copolymer may likewise constitute from about 20 wt. % to about 80 wt. %, in some embodiments from about 30 wt. % to about 70 wt. %, and in some embodiments, from about 40 wt. % to about 60 wt. % of the polymer matrix.

In certain cases, ethylene vinyl acetate copolymer(s) constitute the entire polymer content of the core polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix.

One or more therapeutic agents (e.g., nucleic acid, such as an ASO) are also dispersed within the core polymer matrix that are capable of prohibiting and/or treating a condition, disease, and/or cosmetic state in a patient in need thereof. The therapeutic agent may be prophylactically, therapeutically, and/or cosmetically active, systemically or locally. The therapeutic agent can be homogenously dispersed within the core polymer matrix. Typically, therapeutic agents will constitute from about 5 wt. % to about 80 wt. %, in some embodiments from about 10 wt. % to about 70 wt. %, and in some embodiments, from about 50 wt. % to about 70 wt. % of the core, while the core polymer matrix constitutes from about 40 wt. % to about 95 wt. %, in some embodiments from about 50 wt. % to about 90 wt. %, and in some embodiments, from about 20 wt. % to about 50 wt. % of the core. Suitable therapeutic agents will be further discussed hereinbelow.

The core may also optionally contain one or more excipients if so desired, such as radiocontrast agents, release modifiers, bulking agents, plasticizers, surfactants, crosslinking agents, flow aids, cell permeability enhancers (e.g., fatty acids, such as oleic acid), ribonucleic acid degradation inhibitors (e.g., RNAase and/or DNAse inhibitors), colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the core. In one embodiment, for instance, a radiocontrast agent may be employed to help ensure that the device can be detected in an X-ray based imaging technique (e.g., computed tomography, projectional radiography, fluoroscopy, etc.). Examples of such agents include, for instance, barium-based compounds, iodine-based compounds, zirconium-based compounds (e.g., zirconium dioxide), etc. One particular example of such an agent is barium sulfate. Other known antimicrobial agents and/or preservatives may also be employed to help prevent surface growth and attachment of bacteria, such as metal compounds (e.g., silver, copper, or zinc), metal salts, quaternary ammonium compounds, etc. The core can also be formulated to have a desired flexural modulus of elasticity ranging from about 2 MPa to about 200 MPa.

To help further control the release rate from the implantable medical device, a hydrophilic compound may also be incorporated into the core that is soluble and/or swellable in water. When employed, the weight ratio of the ethylene vinyl acetate copolymer(s) the hydrophilic compounds within the core may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

As noted, cell permeability enhancers may also be employed to help aid in delivery of the nucleic acid. Examples of such permeability enhancers may include, for instance, tight junction modifiers, cyclodextrin, trihydroxy salts (e.g., bile salts, such as sodium glycocholate or sodium fusidate), surfactants (e.g., sodium lauryl sulfate, sodium dodecyl sulfate, cetyltrimethyl ammonium bromide, lauryl betaine, polyoxyethylene sorbitan monopalmitate, etc.), saponin, fusidic acids and derivatives thereof, fatty acids and derivatives thereof (e.g., oleic acid, monoolein, sodium caprate, sodium laurate, etc.), pyrrolidones (e.g., 2-pyrrolidone), alcohols (e.g., ethanol), glycols (e.g., propylene glycol), azones (e.g., laurocapram), terpenes, chelating agents (e.g., EDTA), dendrimers, oxazolidines, diooxolanes (e.g., 2-n-nonyl-1,3-dioxolane), lipids (e.g., phospholipids), and so forth.

To help minimize the risk of nucleic acid degradation, a ribonucleic acid inhibitor may be employed. Representative inhibitors for this purpose may include, for instance, anti-nuclease antibodies and/or non-antibody inhibitors. Suitable nuclease antibodies may be anti-ribonuclease antibodies or anti-deoxyribonuclease antibodies. The anti-ribonuclease antibodies may be antibodies that inhibit one or more of the following ribonucleases or deoxyribonucleases: RNase A, RNase B, RNase C, RNase 1, RNase T1, micrococcal nuclease, S1 nuclease, mammalian ribonuclease 1 family, ribonuclease 2 family, mammalian angiogenins, RNase H family, RNase L, eosinophil RNase, messenger RNA ribonucleases (5′-3′ Exoribonucleases, 3′-5′ Exoribonucleases), decapping enzymes, deadenylases, E. coli endoribonucleases (RNase P, RNase III, RNase E, RNase 1,1*, RNase HI, RNase HII, RNase M, RNase R, RNase IV, F; RNase P2,0, PIV, PC, RNase N), E. coli exoribonucleases (RNase II, PNPase, RNase D, RNase BN, RNase T, RNase PH, OligoRNase, RNase R), RNase Sa, RNase F1, RNase U2, RNase Ms, RNase St, DNase 1, S1 nuclease, and micrococcal nuclease. Suitable non-antibody nuclease inhibitors may likewise include, but are not limited to, diethyl pyrocarbonate, ethanol, formamide, guanidinium thiocyanate, vanadyl-ribonucleoside complexes, macaloid, sodium dodecylsulfate (SDS), proteinase K, heparin, hydroxylamine-oxygen-cupric ion, bentonite, ammonium sulfate, dithiothreitol (DTT), β-mercaptoethanol, cysteine, dithioerythritol, urea, polyamines (spermidine, spermine), detergents (e.g., sodium dodecylsulfate), tris (2-carboxyethyl) phosphene hydrochloride (TCEP), and so forth. Chelating agents are also suitable non-antibody nuclease inhibitors as such compounds can help bind cations (e.g., calcium, iron, etc.) that would otherwise cause degradation. The chelating agent may include, for instance, aminocarboxylic acids (e.g., ethylenediaminetetraacetic acid) and salts thereof, hydroxycarboxylic acids (e.g., citric acid, tartaric acid, ascorbic acid, etc.) and salts thereof, polyphosphoric acids (e.g., tripolyphosphoric acid, hexametaphosphoric acid, etc.) and salts thereof, and so forth. Desirably, the chelating agent is multidentate in that it is capable of forming multiple coordination bonds with metal ions to reduce the likelihood that any of the free metal ions. In one embodiment, for example, a multidentate chelating agent containing two or more aminodiacetic (sometimes referred to as iminodiacetic) acid groups or salts thereof may be utilized. One example of such a chelating agent is ethylenediaminetetraacetic acid (EDTA). Examples of suitable EDTA salts include calcium disodium EDTA, diammonium EDTA, disodium and dipotassium EDTA, trisodium and tripotassium EDTA, tetrasodium and tetrapotassium EDTA. Still other examples of similar aminodiacetic acid chelating agents include, but are not limited to, butylenediaminetetraacetic acid, (1,2-cyclohexylenediaminetetraacetic acid (CyDTA), diethylenetriaminepentaacetic acid (DTPA), ethylenediaminetetrapropionic acid, (hydroxyethyl)ethylenediaminetriacetic acid (HEDTA), triethanolamine EDTA, triethylenetetraminehexaacetic acid (TTHA), 1,3-diamino-2-hydroxypropane-N,N,N′,N′-tetraacetic acid (DHPTA), methyliminodiacetic acid, propylenediaminetetraacetic acid, ethylenediiminodipropanedioic acid (EDDM), 2,2′-bis(carboxymethyl)iminodiacetic acid (ISA), ethylenediiminodibutandioic acid (EDDS), and so forth. Still other suitable multidentate chelating agents include N,N,N′,N′-ethylenediaminetetra(methylenephosphonic)acid (EDTMP), nitrilotrimethyl phosphonic acid, 2-aminoethyl dihydrogen phosphate, 2,3-dicarboxypropane-1,1-diphosphonic acid, meso-oxybis(butandionic acid) (ODS), and so forth.

One or more nonionic, anionic, and/or amphoteric surfactants may also be employed to help create a uniform dispersion. When employed, such surfactant(s) typically constitute from about 0.05 wt. % to about 8 wt. %, and in some embodiments, from about 0.1 wt. % to about 6 wt. %, and in some embodiments, from about 0.5 wt. % to about 3 wt. % of the membrane layer. Nonionic surfactants, which typically have a hydrophobic base (e.g., long chain alkyl group or an alkylated aryl group) and a hydrophilic chain (e.g., chain containing ethoxy and/or propoxy moieties), are particularly suitable. Some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. Particularly suitable nonionic surfactants may include ethylene oxide condensates of fatty alcohols, polyoxyethylene ethers of fatty acids, polyoxyethylene sorbitan fatty acid esters, and sorbitan fatty acid esters, etc. The fatty components used to form such emulsifiers may be saturated or unsaturated, substituted or unsubstituted, and may contain from 6 to 22 carbon atoms, in some embodiments from 8 to 18 carbon atoms, and in some embodiments, from 12 to 14 carbon atoms. Sorbitan fatty acid esters (e.g., monoesters, diester, triesters, etc.) that have been modified with polyoxyethylene are one particularly useful group of nonionic surfactants. These materials are typically prepared through the addition of ethylene oxide to a 1,4-sorbitan ester. The addition of polyoxyethylene converts the lipophilic sorbitan ester surfactant to a hydrophilic surfactant that is generally soluble or dispersible in water. Such materials are commercially available under the designation TWEEN® (e.g., TWEEN® 80, or polyethylene (20) sorbitan monooleate).

### II. Therapeutic Agents

As indicated herein a therapeutic agent (e.g., a nucleic acid) can be dispersed within the core, such as throughout the core polymer matrix. As used herein, the term “nucleic acid” generally refers to a compound comprising a nucleobase and an acidic moiety, e.g., a nucleoside, nucleotide, polynucleotide, or a combination thereof. A “nucleoside” generally refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” generally refers to a nucleoside including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides. For example, polynucleotides may contain three or more nucleotides as linear molecules, in which adjacent nucleotides are linked to each other via a phosphodiester linkage. The term “nucleic acid” also encompasses RNA as well as single and/or double-stranded DNA. More particularly nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-c-LNA having a 2′-amino functionalization), ethylene nucleic acids (ENA), cyclohexenyl nucleic acids (CeNA) or chimeras or combinations thereof.

Nucleic acids may be naturally occurring, for example, in the context of a genome, a transcript, a mRNA, tRNA, rRNA, siRNA, snRNA, plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Nucleic acids can be purified from natural sources, produced using recombinant expression systems and optionally purified, chemically synthesized, etc. The nucleic acids may also include nucleoside analogs, such as analogs having chemically modified bases or sugars, and backbone modifications. In some embodiments, the nucleic acid is or contains natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Modified nucleotide base pairing may be employed and encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

In certain embodiments, the nucleic acid may be a polynucleotide (e.g., RNA polynucleotides, such as mRNA polynucleotides) in which one or more nucleobases have been modified for therapeutic purposes. In fact, in certain embodiments, a polynucleotide (e.g., RNA polynucleotide, such as mRNA polynucleotide) may be employed that includes a combination of at least two (e.g., 2, 3, 4 or more) modified nucleobases. For example, suitable modified nucleobases in the polynucleotide may be a modified cytosine, such as 5-methylcytosine, 5-methyl-cytidine (m5C), N4-acetyl-cytidine (ac4C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine, etc.; modified uridine, such as 5-cyano uridine, 4′-thio uridine, pseudouridine (L), N1-methylpseudouridine (m1Lp), N1-ethylpseudouridine, 2-thiouridine (s2U), 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine (mo5U), 5-methoxyuridine, 2′-O-methyl uridine, etc.; modifined guanosine, such as α-thio-guanosine, inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1 G), 8-oxo-guanosine, 7-methyl-8-oxo-guanosine, etc.; modified adenine, such as α-thio-adenosine, 7-deaza-adenine, 1-methyl-adenosine (m1A), 2-methyl-adenine (m2A), N6-methyl-adenosine (m6A), 2,6-diaminopurine, etc.; as well as combinations thereof. In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the polynucleotide (e.g., RNA polynucleotide, such as mRNA polynucleotide) may be uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as any of those set forth above.

In some embodiments, polynucleotides function as messenger RNA (mRNA). “Messenger RNA” (mRNA) generally refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The basic components of a mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features that serve to overcome existing problems of effective polypeptide expression using nucleic-acid based therapeutics. The mRNA may contain at least one (one or more) ribonucleic acid (RNA) polynucleotide having an open reading frame encoding at least one polypeptide of interest. In some embodiments, a RNA polynucleotide of a mRNA encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 polypeptides. In some embodiments, an RNA polynucleotide of a mRNA encodes at least 10, 20, 30, 40, 50, 60, 70, 80, 90 or 100 polypeptides. In some embodiments, a RNA polynucleotide of a mRNA encodes at least 100 or at least 200 polypeptides.

In some embodiments, the nucleic acids are therapeutic mRNAs. As used herein, the term “therapeutic mRNA” refers to a mRNA that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of various diseases and conditions, such as bacterial infections, viral infections, parasitic infections, cell proliferation disorders, genetic disorders, and autoimmune disorders. The mRNA may be designed to encode polypeptides of interest selected from any of several target categories including, but not limited to, biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, targeting moieties or those proteins encoded by the human genome for which no therapeutic indication has been identified but which nonetheless have utility in areas of research and discovery.

Particularly suitable therapeutic mRNAs are those that include at least one ribonucleic acid (RNA) polynucleotide having an open reading frame encoding at least one antigenic polypeptide, in which the RNA polynucleotide of the RNA includes at least one chemical modification. The chemical modification may, for instance, be pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine), 5-methoxyuridine, and 2′-O-methyl uridine.

Although by no means required, the particular nature of the nucleic acid may also be selected to help improve its ability to be dispersed within the polymer matrix and delivered to a patient without significant degradation. For instance, it may be desired to co-deliver a conventional RNA (e.g., mRNA) with a self-amplifying RNA. Conventional mRNAs, for instance, generally include an open reading frame for the target antigen, flanked by untranslated regions and with a terminal poly(A) tail. After transfection, they drive transient antigen expression. Self-amplifying mRNAs, on the other hand, are capable of directing their self-replication, through synthesis of the RNA-dependent RNA polymerase complex, generating multiple copies of the antigen-encoding mRNA, and express high levels of the heterologous gene when they are introduced into the cytoplasm of host cells. Circular RNA (circRNA), which is a single-stranded RNA joined head to tail, may also be employed. The target RNA may be circularized, for example, by backsplicing of a non-mammalian exogenous intron or splint ligation of the 5′ and 3′ ends of a linear RNA. Examples of suitable circRNAs are described, for instance, in U.S. Patent Publication No. 2019/0345503, which is incorporated herein by reference thereto.

Antisense RNA (e.g., ASOs) may also be employed, which generally has a base carried on a backbone subunit, containing one or more backbone groups (e.g., phosphorothioates or morpholino backbone groups) and in which the backbone groups are linked by inter-subunit linkages (both charged and uncharged) that allow the bases in the compound to hybridize to a target sequence in an RNA by Watson-Crick base pairing, thereby forming an RNA:oligonucleotide heteroduplex within the target sequence. ASOs are complementary to a section of naturally occurring RNA, such as mRNA or viral RNA, to form Watson-Crick base pairs and thus to inhibit a biological function of the RNA. As used herein “antisense oligonucleotide” refers to a nucleotide sequence which is substantially complementary to a target nucleotide sequence in a pre-mRNA molecule, hrRNA (heterogenous nuclear RNA), or mRNA molecule. The degree of complementarity (or substantial complementarity) of the antisense sequence is preferably such that a molecule comprising the antisense sequence can form a stable hybrid with the target nucleotide sequence in the RNA molecule under physiological conditions.

The length of the ASO can range from about 5 nucleotide or nucleoside subunits in length to about 50 nucleotide or nucleoside subunits in length. As will be appreciated, a subunit is a nucleobase and a sugar combination (e.g., ribose or deoxyribose) suitably bound to adjacent subunits through phosphorus or non-phosphorus linkages, as further discussed hereinbelow. The length of the ASO can range from about 10 subunits to about 45 subunits, such as from about 15 subunits to about 40 subunits, such as from about 20 subunits to about 35 subunits. In certain embodiments, the ASO has a length of from about 10 subunits to about 25 subunits. The length of the ASO can vary so long as it is capable of binding selectively to the intended location on an RNA target.

Natural, or unmodified, oligonucleotides are susceptible to nuclease degradation and poor protein binding. Accordingly, unmodified oligonucleotides have inefficient tissue uptake precluding their use as clinically effective therapeutic agents. Modifications to the oligonucleotides, however, have been shown to decrease nuclease degradation. For example, multiple types of modifications to internucleoside linkages in nucleotides or nucleosides can improve various properties of the ASO. Chemical modifications to the phosphodiester backbone can be made in order to improve pharmacokinetic properties, tolerability profile, and/or target binding affinity. For instance, certain modifications to the phosphodiester backbone that can be utilized in ASOs of the present disclosure include those as shown in (b)-(j) below. Notably, (a) illustrates an unmodified DNA oligonucleotide (ODN) while (b)-(j) illustrate suitable modifications to the internucleoside linkages as follows: (b) phosphorothioate ODN (PS-ODN), (c) R-isomer of PS-ODN, (Rp-PS-ODN), (d) S-isomer of PS-ODN, (Sp-PS-ODN), (e) methylphosphonate ODN (PM-ODN), (f) phosphoramidate ODN (PN-ODN) where R=H or alkyl, (g) phosphomopholidate, (h) phosphopiperazidate, (i) phosphorodiamidate morpholino (PMO), and (j) peptide nucleic acids (PNA), where B=nucleobases, including adenine (A), guanine (G), cytosine (C), thymine (T), or uracil (U). ASOs of the present disclosure can also include tricyclo-DNAs (tcDNA) subunits as part of the oligomer.

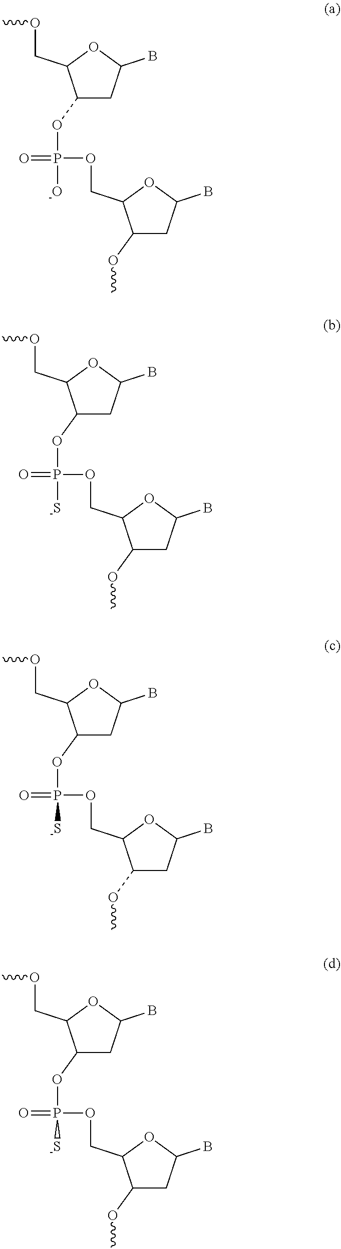

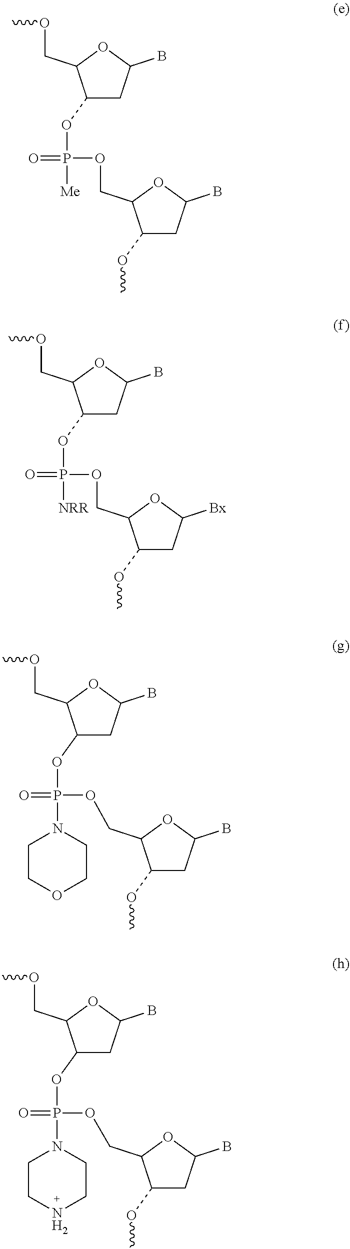

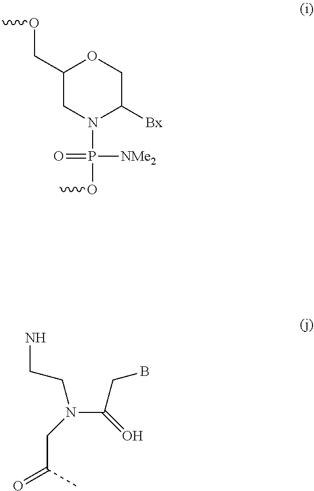

Any of the chemical modifications as shown in (b)-(j) or otherwise described herein can be incorporated into ASOs of the present disclosure. Notably, the ASO can include a nucleoside having one or more chemically modified internucleoside linkages. As used herein “chemically modified” when referring to internucleoside linkages includes any of the chemical modifications provided hereinabove with respect to (b)-(j). In certain embodiments, at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 65%, such as at least 70%, such as at least 75%, such as at least 80%, such as at least 85%, such as at least 90%, such as at least 95%, such as at least 100%, of the internucleoside linkages in the ASO are chemically modified. In other embodiments, chemically modified internucleoside linkages can be disposed about the 3′ end or the 5′ end of the ASO. For instance, at least one and up to five of the internucleoside linkages extending from either the 3′ terminus or the 5′ terminus can include chemically modified internucleoside linkages. In certain embodiments, from about 50% to about 100% of the internucleoside linkages are phosphorothioate linkages (shown in (b), (c), and (d) above). In other embodiments, from about 50% to about 100% of the internucleoside linkages are morpholino subunits (shown in (i) above). Morpholino oligonucleotides with uncharged backbone linkages are detailed, for example, in U.S. Pat. Nos. 5,698,685, 5,217,866, 5,142,047, 5,034,506, 5,166,315, 5,185,444, 5,521,063, and 5,506,337, which are incorporated herein by reference. Other exemplary antisense oligonucleotides are described in U.S. Pat. Nos. 9,464,292, 10,131,910, 10,144,762, and 10,913,947, which are incorporated herein by reference.

Modifications to the 2′ position of the sugar moiety of the nucleoside subunit is also contemplated. For example, the 2′-position on the sugar moiety can be substituted with a 2′-substituent group. Such 2′-substituent groups can include 2′-fluoro, 2′-alkoxy, 2′-aminoalkoxy, 2′-allyloxy, 2′-imidazole-alkoxy and 2′-poly(ethylene oxide). Alkoxy and aminoalkoxy groups generally include lower alkyl groups, particularly C1-C9 alkyl. Poly(ethylene glycols) are of the structure (O—CH2-CH2)n-O-alkyl. Other 2′-substituent groups can include 2′-O-methyl (2′-OMe), 2′-O-methoxyethyl (2′-MOE) and locked nucleic acid (LNA) as shown in (k)-(m) below, where base=heterocyclic bases.

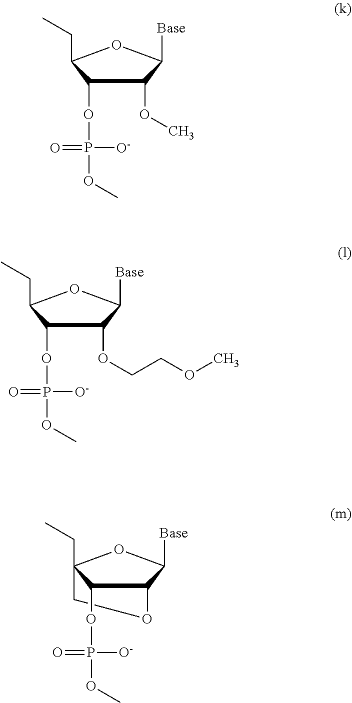

The use of 2′-substituent groups can increase the binding affinity of the substituted oligonucleotides or can prevent nuclease degradation in vivo. Notably, at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 65%, such as at least 70%, such as at least 75%, such as at least 80%, such as at least 85%, such as at least 90%, such as at least 95%, such as at least 100% of nucleotides in the ASO can include modified 2′-substituent groups.

The ASOs of the present disclosure can also include modified bases in the nucleotide units that make up the oligonucleotides of the ASO. Such modified bases may include 6-azapyrimidines and N-2, N-6 and O-6 substituted purines including 2-aminopropyladenine. Other modified pyrimidine and purine base are expected to increase the binding affinity of oligonucleotides to a complementary strand of nucleic acid.

Notably, the ASOs of the present disclosure can include “first generation” ASOs, which refer to oligodeoxynucleotides having 2′-deoxy ribonucleotides and phosphorothioate internucleoside linkages. Suitable ASOs can also include ASOs that are oligonucleotides having a 2′-deoxy “gap” region flanked by “wings” having nucleotides with 2′-modified ribonucleotides, referred to as “gapmers”. Suitable second generation ASOs include an “MOE gapmer” in which the 2′-modified ribonucleotide is a 2′-O-methoxyethyl (2′-MOE or simply MOE) modification, and each of the internucleoside linkages is a phosphorothioate. Second generation ASOs may have a length of 20 nucleotides of which the 5 nucleotides at each terminus are 2′-MOE nucleotides and the center ten nucleotides are 2′-deoxyribonucleotides. Accordingly, these second generation ASOs are referred to as “5-10-5 MOE gapmers” since they have a 5-10-5 wing-gap-wing motif. Suitable 5-10-5 gapmers can have the following formula:

5′ mB-mB-mB-mB-mB-dB-dB-dB-dB-dB-dB-dB-dB-dB-dB-mB-mB-mB-mB-mB 3′

where mB=2′-O-methoxyethyl ribonucleoside; dB=deoxynucleoside; “-”=phosphorothiate linkage, and b=heterocyclic base.

One example 5-10-5 gapmer that can be used as an ASO for the present disclosure is shown in SEQ. ID. NO. 1. The ASO can include a gap region having at least 5 to about 15, such as about 8 to about 10, contiguous 2′-deoxyribonucleosides and a first wing region and a second wing region flaking the gap region. Each of the first and second wing regions independently can include 1 to about 8 2′-O-(2-methoxyethyl) ribonucleotides. The ASOs of the present disclosure can also include “hemimers,” which are chimeric compounds in which there is a single 2′-modified “wing” adjacent to (on either the 5′, or the 3′ side of) a 2′-deoxy gap. The wing present in the hemimer can include 2 nucleotides to about 8 nucleotides. Notably, the gap region, first wing region, or second wing region, can include any of the internucleoside chemical modifications or 2′-substituent groups as disclosed herein.

The ASOs of the present disclosure can also include ASO conjugates, where one or more moieties are conjugated to the ASO. For instance, attached moieties can include peptides, proteins, carbohydrates, fatty acids, aptamers, and small molecules including cholesterol, tocopherol, and folic acid. Specifically, the ASO can also be conjugated to anisamide, stearic acid, cyclic RGD peptide, anandamide, N-acetylgalactosamine, spermine, and combinations thereof. For ASO conjugates, the selected moiety can be conjugated to the ASO in a variety of locations, however, in certain embodiments, the selected moiety is tethered to the 3′-terminus or the 5′-terminus. Different linkers can be used to conjugate the moiety to the ASO. Suitable linkers can include thiol linkers, amino linkers, aldehyde linkers, azide linkers, carboxyl linkers, dibenzylcyclooctyne linkers. ASOs (e.g., morpholino oligonucleotides) can be conjugated to arginine-rich cell penetrating peptides (CPPs). ASOs can also be conjugated to polyethylene glycol (PEG) moieties. The ASO conjugates can include saccharides, such as the monosaccharide GaINAc. For instance, up to three GalNac molecules can be attached to the ASO via a tridentate linker to the 3′-terminus of the ASO. These moieties can be attached to the ASO to improve uptake mechanisms and pharmacokinetic properties of the ASO. In certain embodiments, the ASO can include a dynamic polyconjugate, which is a macromolecule having an ASO that undergoes structural modification in vivo to yield the ASO. Suitable ASO conjugates are further described in U.S. 2016/0289677, WO 2004/044141, WO 2009/073809, WO 2012/083046, WO 2012/089352, WO 2012/089602, WO 2005/086775, WO 2011/126937, WO 2009/025669, which are incorporated by reference herein.

In certain embodiments, the ASO can include an oligonucleotide that has been approved by the U.S. Food and Drug Administration (FDA). Certain approved oligonucleotides include Vitravene™ (Formiversen), Macugen™ (Pegaptanib), Kynamro™ (Mipomersen), Defitelio™ (Defibrotide), Exondys 51™ (Eteplirsen), Spinraza™ (Nusinersen), Tegsedi™ (Inotersen), Onpattro™ (Patisiran), Waylivra™ (Volanesoren), Givlaari™ (Givosiran), Vyondys 53 (Golodirsen), Viltepso™ (Vitolarsen), Oxlumo™ (Lumasiran), Leqvio™ (Inclisiran), Amondys 45™ (Casimersen), and combinations thereof. Other ASOs can include Camlingo™ (Alicaforsen), Drisapersen (PR0051, GSK-2402968), Custirsen (OGX-011 and CC-8490), Miraversen (SPC3649), ISIS-TTR02, Aezea™ (Cenersen), GTI-2040, Imetelstat (GRN163L), ISIS-STAT3RX, Liposomal Grb-2 (BP-100-1.01), and combinations thereof.

In certain cases, the nucleic acid may be an aptamer, such as an RNA aptamer. An RNA aptamer may be any suitable RNA molecule that can be used on its own as a stand-alone molecule or may be integrated as part of a larger RNA molecule having multiple functions, such as an RNA interference molecule. For example, an RNA aptamer may be located in an exposed region of a shRNA molecule (e.g., the loop region of the shRNA molecule) to allow the shRNA or miRNA molecule to bind a surface receptor on the target cell. After it is internalized, it may then be processed by the RNA interference pathways of the target cell. The nucleic acid that forms the nucleic acid aptamer may include naturally occurring nucleosides, modified nucleosides, naturally occurring nucleosides with hydrocarbon linkers (e.g., an alkylene), and/or or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides of the nucleic acid aptamer can be replaced with a hydrocarbon linker or a polyether linker. Suitable aptamers may be described, for instance, in U.S. Pat. No. 9,464,293, which is incorporated herein by reference thereto.

Protein-fused nucleic acids may also be suitable for use in the present disclosure. For example, proteins (e.g., antibodies) may be covalently linked to RNA (e.g., mRNA). Such RNA-protein fusions may be synthesized by in vitro or in situ translation of mRNA pools containing a peptide acceptor attached to their 3′ ends. In one embodiment, after readthrough of the open reading frame of the message, the ribosome pauses when it reaches the designed pause site, and the acceptor moiety occupies the ribosomal A site and accepts the nascent peptide chain from the peptidyl-tRNA in the P site to generate the RNA-protein fusion. The covalent link between the protein and the RNA (in the form of an amide bond between the 3′ end of the mRNA and the C-terminus of the protein that it encodes) allows the genetic information in the protein to be recovered and amplified (e.g., by PCR) following selection by reverse transcription of the RNA. Once the fusion is generated, selection or enrichment is carried out based on the properties of the mRNA-protein fusion, or, alternatively, reverse transcription may be carried out using the mRNA template while it is attached to the protein to avoid the impact of the single-stranded RNA on the selection. Examples of such protein-fused nucleic acids are described, for instance, in U.S. Pat. No. 6,518,018, which is incorporated herein by reference. Ribozymes (e.g., DNAzyme and/or RNAzyme) may also be employed that are conjugated to nucleic acids having a sequence that catalytically cleaves RNA, such as described in U.S. Pat. No. 10,155,946, which is incorporated herein by reference.

Apart from single strand nucleic acids such as described above, various specific types of double strand nucleic acids may also be employed to help improve stability. Circular DNA (cDNA) and plasmid nucleic acids (e.g., pDNA), which are a closed circular form of DNA, may be employed in certain embodiments. Examples of such nucleic acids are described, for instance, in WO 2004/060277 which is incorporated herein by reference. Long double stranded DNA may also be employed. For instance, a scaffolded DNA origami may be employed in which the long single-stranded DNA is folded into a certain shape by annealing the scaffold in the presence of shorter oligonucleotides (“staples”) containing segments or regions of complementary sequences to the scaffold. Examples of such structures are described, for instance, in U.S. Patent Publication Nos. 2019/0142882 and 2018/0171386, which are incorporated herein by reference.

### III. Device Configuration

### A. Core

As noted above, a core may be formed from the polymer matrix, nucleic acid, and optional excipients. The core and/or implantable medical device may have a variety of different geometric shapes, such as cylindrical (rod), disc, ring, doughnut, helical, elliptical, triangular, ovular, etc. In one embodiment, for example, the core and/or implantable medical device may have a generally circular cross-sectional shape so that the overall structure is in the form of a cylinder (rod) or disc. In such embodiments, the core and/or implantable medical device will typically have a diameter of from about 0.5 to about 50 millimeters, in some embodiments from about 1 to about 40 millimeters, and in some embodiments, from about 5 to about 30 millimeters. The length of the core and/or implantable medical device may vary, but is typically in the range of from about 1 to about 25 millimeters. Cylindrical devices may, for instance, have a length of from about 5 to about 50 millimeters, while disc-shaped devices may have a length of from about 0.5 to about 5 millimeters.

The core may be formed through a variety of known techniques, such as by hot-melt extrusion, injection molding, solvent casting, dip coating, spray coating, microextrusion, coacervation, etc. In one embodiment, a hot-melt extrusion technique may be employed. Hot-melt extrusion is generally a solvent-free process in which the components of the core (e.g., ethylene vinyl acetate copolymer(s), nucleic acid(s), optional excipients, etc.) may be melt blended and optionally shaped in a continuous manufacturing process to enable consistent output quality at high throughput rates. This technique is particularly well suited to ethylene vinyl acetate copolymers as they typically exhibit a relatively high degree of long-chain branching with a broad molecular weight distribution. This combination of traits can lead to shear thinning of the copolymer during the extrusion process, which help facilitates hot-melt extrusion. Furthermore, the polar vinyl acetate comonomer units can serve as an “internal” plasticizer by inhibiting crystallization of the polyethylene chain segments. This may lead to a lower melting point of the copolymer, which further enhances its ability to be processed with the nucleic acid.

During a hot-melt extrusion process, melt blending generally occurs at a temperature that is similar to or slightly above the melting temperature of the ethylene vinyl acetate copolymer(s). The melt blending temperature may, for example, be from about 30° C. to about 100° C., in some embodiments, from about 40° C. to about 80° C., and in some embodiments, from about 50° C. to about 70° C. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical, and it may contain any number and/or orientation of threads and channels as is known in the art. For example, the screw typically contains a thread that forms a generally helical channel radially extending around the center of the screw. A feed section and melt section may be defined along the length of the screw. The feed section is the input portion of the barrel where the ethylene vinyl acetate copolymer(s) and/or nucleic acid are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows. As noted, the extruder can have multiple sections (e.g., mixing, melting, etc.) that can be operated at different temperatures depending on the polymer and nucleic acid utilized. For instance, in embodiments, the extruder can define at least two, such as at least three, such as at least four, such as at least five, such as at least six, such as at least seven, such as at least eight, such as at least nine, such as at least 10 distinct zones, each can be configured to operate at a different temperature, if desired. Temperatures of the zones can range from about 40° C. to about 75° C., such as from about 50° C. to about 65° C.

Once melt blended together, the resulting polymer composition may be extruded through an orifice (e.g., die) and formed into pellets, sheets, fibers, filaments, etc., which may be thereafter shaped into a core using a variety of known shaping techniques, such as injection molding, compression molding (e.g., vacuum compression molding), nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present disclosure. In one embodiment, an injection molding apparatus may be employed that includes a first mold base and a second mold base, which together define a mold cavity having the shape of the core. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape for the core (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 50° C. to about 120° C., in some embodiments from about 60° C. to about 110° C., and in some embodiments, from about 70° C. to about 90° C.

As indicated above, another suitable technique for forming a core of the desired shape and size is three-dimensional printing. During this process, the polymer composition may be incorporated into a printer cartridge that is readily adapted for use with a printer system. The printer cartridge may, for example, contains a spool or other similar device that carries the polymer composition. When supplied in the form of filaments, for example, the spool may have a generally cylindrical rim about which the filaments are wound. The spool may likewise define a bore or spindle that allows it to be readily mounted to the printer during use. Any of a variety of three-dimensional printer systems can be employed in the present disclosure. Particularly suitable printer systems are extrusion-based systems, which are often referred to as “fused deposition modeling” systems. For example, the polymer composition may be supplied to a build chamber of a print head that contains a platen and gantry. The platen may move along a vertical z-axis based on signals provided from a computer-operated controller. The gantry is a guide rail system that may be configured to move the print head in a horizontal x-y plane within the build chamber based on signals provided from controller. The print head is supported by the gantry and is configured for printing the build structure on the platen in a layer-by-layer manner, based on signals provided from the controller. For example, the print head may be a dual-tip extrusion head.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50° C. to about 120° C., in some embodiments from about 60° C. to about 110° C., and in some embodiments, from about 70° C. to about 90° C. A vacuum source may also apply a negative pressure to the precursor during molding to help ensure that it retains a precise shape. Examples of such compression molding techniques are described, for instance, in U.S. Pat. No. 10,625,444 to Treffer, et al., which is incorporated herein in its entirety by reference thereto.

### B. Membrane Layer

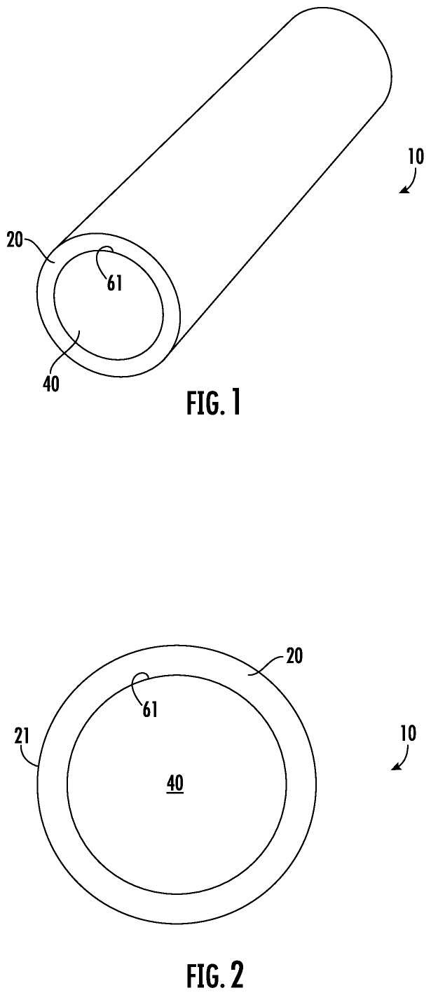

In some cases, the implantable medical device may be multilayered in that it contains at least one membrane layer positioned adjacent to an outer surface of the core. The number of membrane layers may vary depending on the particular configuration of the device, the nature of the nucleic acid, and the desired release profile. For example, the device may contain only one membrane layer. Referring to FIGS. 1-2, for example, one embodiment of an implantable medical device 10 is shown that contains a core 40 having a generally circular cross-sectional shape and is elongated so that the resulting device is generally cylindrical in nature. The core 40 defines an outer circumferential surface 61 about which a membrane layer 20 is circumferentially disposed. Similar to the core 40, the membrane layer 20 also has a generally circular cross-sectional shape and is elongated so that it covers the entire length of the core 40. During use of the device 10, a nucleic acid is capable of being released from the core 40 and through the membrane layer 20 so that it exits from an external surface 21 of the device.

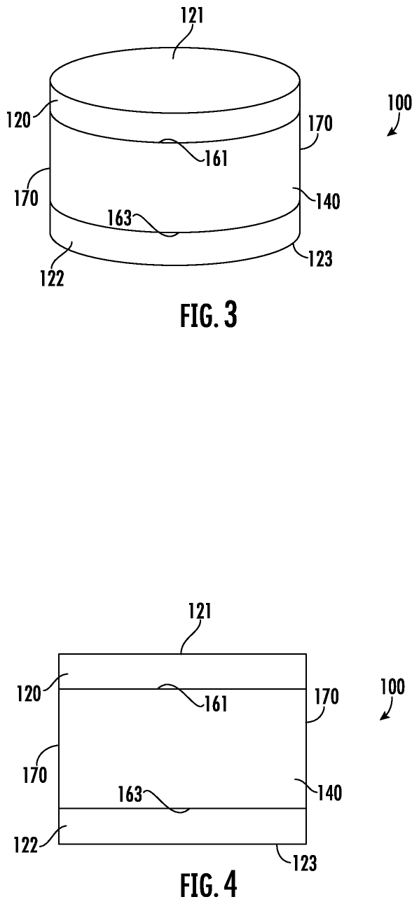

Of course, in other embodiments, the device may contain multiple membrane layers. In the device of FIGS. 1-2, for example, one or more additional membrane layers (not shown) may be disposed over the membrane layer 20 to help further control release of the nucleic acid. In other embodiments, the device may be configured so that the core is positioned or sandwiched between separate membrane layers. Referring to FIGS. 3-4, for example, one embodiment of an implantable medical device 100 is shown that contains a core 140 having a generally circular cross-sectional shape and is elongated so that the resulting device is generally disc-shaped in nature. The core 140 defines an upper outer surface 161 on which is positioned a first membrane layer 120 and a lower outer surface 163 on which is positioned a second membrane layer 122. Similar to the core 140, the first membrane layer 120 and the second membrane layer 122 also have a generally circular cross-sectional shape that generally covers the core 140. If desired, edges of the membrane layers 120 and 122 may also extend beyond the periphery of the core 140 so that they can be sealed together to cover any exposed areas of an external circumferential surface 170 of the core 140. During use of the device 100, a nucleic acid is capable of being released from the core 140 and through the first membrane layer 120 and second membrane layer 122 so that it exits from external surfaces 121 and 123 of the device. Of course, if desired, one or more additional membrane layers (not shown) may also be disposed over the first membrane layer 120 and/or second membrane layer 122 to help further control release of the nucleic acid.

Regardless of the particular configuration employed, the membrane layer(s) generally contain a membrane polymer matrix that contains a hydrophobic polymer. The membrane polymer matrix typically constitutes from about 30 wt. % to 100 wt. %, in some embodiments, from about 40 wt. % to about 99 wt. %, and in some embodiments, from about 50 wt. % to about 90 wt. % of a membrane layer. When employing multiple membrane layers, it is typically desired that each membrane layer contains a membrane polymer matrix that includes such a hydrophobic polymer. For example, a first membrane layer may contain a first membrane polymer matrix and a second membrane layer may contain a second membrane polymer matrix. In such embodiments, the first and second membrane polymer matrices each contain a hydrophobic polymer, which may be the same or different.

The polymer(s) used in the membrane polymer matrix are generally hydrophobic in nature so that they can retain its structural integrity for a certain period of time when placed in an aqueous environment, such as the body of a mammal, and stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also suitable for use in the membrane polymer matrix. In certain embodiments, the membrane polymer matrix may contain a semi-crystalline olefin copolymer. The melting temperature of such an olefin copolymer may, for instance, range from about 20° C. to about 100° C., in some embodiments from about 25° C. to about 80° C., in some embodiments from about 30° C. to about 70° C., in some embodiments from about 35° C. to about 65° C., and in some embodiments, from about 40° C. to about 60° C., such as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, the present inventors have discovered that certain aspects of the copolymer can be selectively controlled to help achieve the desired release properties. For instance, the polar monomeric content of the copolymer may be selectively controlled to be within a range of from about 20 wt. % to about 60 wt. %, in some embodiments from about 25 wt. % to about 55 wt. %, in some embodiments from about 30 wt. % to about 50 wt. %, in some embodiments from about 35 wt. % to about 48 wt. %, and in some embodiments, from about 38 wt. % to about 45 wt. % of the copolymer. Conversely, the olefin monomeric content of the copolymer may likewise be within a range of from about 40 wt. % to about 80 wt. %, 45 wt. % to about 75 wt. %, in some embodiments from about 50 wt. % to about 80 wt. %, in some embodiments from about 52 wt. % to about 65 wt. %, and in some embodiments, from about 55 wt. % to about 62 wt. %.

The hydrophobic polymer used in the membrane polymer matrix may also be the same or different than the ethylene vinyl acetate copolymer(s) employed in the core. In one embodiment, for instance, both the core and the membrane layer(s) employ the same polymer (e.g., ethylene vinyl acetate copolymer). In yet other embodiments, the membrane layer(s) may employ a hydrophobic polymer (e.g., α-olefin copolymer) that has a lower melt flow index than the ethylene vinyl acetate copolymer employed in the core. Among other things, this can further help control the release of the nucleic acid from the device. For example, the ratio of the melt flow index of a ethylene vinyl acetate copolymer employed in the core to the melt flow index of a hydrophobic polymer employed in the membrane layer(s) may be from about 1 to about 20, in some embodiments about 2 to about 15, and in some embodiments, from about 4 to about 12. The melt flow index of the hydrophobic polymer in the membrane layer(s) may, for example, range from about 1 to about 80 g/10 min, in some embodiments from about 2 to about 70 g/10 min, and in some embodiments, from about 5 to about 60 g/10 min, as determined in accordance with ASTM D1238-13 at a temperature of 190° C. and a load of 2.16 kilograms. Examples of suitable ethylene vinyl acetate copolymers that may be employed include those available from Celanese under the designation ATEVA® (e.g., ATEVA® 4030AC or 2861A).

The membrane layer(s) used in the device may optionally contain a nucleic acid, such as described above, which are dispersed within the membrane polymer matrix. The nucleic acid in the membrane layer(s) may be the same or different than those employed in the core. Regardless, when a nucleic acid is employed in a membrane layer, it is generally desired that the membrane layer generally contains the nucleic acid in an amount such that the ratio of the concentration (wt. %) of the nucleic acid in the core to the concentration (wt. %) of the nucleic acid in the membrane layer is greater than 1, in some embodiments about 1.5 or more, and in some embodiments, from about 1.8 to about 4. When employed, nucleic acids typically constitute only from about 1 wt. % to about 40 wt. %, in some embodiments from about 5 wt. % to about 35 wt. %, and in some embodiments, from about 10 wt. % to about 30 wt. % of a membrane layer. Of course, in other embodiments, the membrane layer is generally free of a nucleic acid prior to release from the core. When multiple membrane layers are employed, each membrane layer may generally contain the nucleic acid in an amount such that the ratio of the weight percentage of the nucleic acid in the core to the weight percentage of the nucleic acid in the membrane layer is greater than 1, in some embodiments about 1.5 or more, and in some embodiments, from about 1.8 to about 4.

The membrane layer(s) may also optionally contain one or more excipients as described above, such as radiocontrast agents, hydrophilic compounds, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

To help further control the release rate from the implantable medical device, for example, a hydrophilic compound may also be incorporated into the membrane layer such as described above. When employed, the weight ratio of the hydrophobic polymers to the hydrophilic compounds within the membrane layer may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 50 wt. %, in some embodiments from about 2 wt. % to about 40 wt. %, and in some embodiments, from about 5 wt. % to about 30 wt. % of the membrane layer, while hydrophobic polymers typically constitute from about 50 wt. % to about 99 wt. %, in some embodiments from about 60 wt. % to about 98 wt. %, and in some embodiments, from about 70 wt. % to about 95 wt. % of the membrane layer.

In one particular embodiment, the membrane layer(s) may contain a hydrophilic compound that is in the form of a plurality of water-soluble particles distributed within a membrane polymer matrix. In such embodiments, the particle size of the water-soluble particles may be controlled to help achieve the desired delivery rate. More particularly, the median diameter (D50) of the particles may be about 150 micrometers or less, in some embodiments 125 micrometers or less, in some embodiments 100 micrometers or less, in some embodiments about 80 micrometers or less, in some embodiments about 60 micrometers or less, and in some embodiments, from about 1 to about 40 micrometers, such as 25 micrometers. The median diameter of the water-soluble particles can be determined using a laser scattering particle size distribution analyzer (e.g., LA-960 from Horiba). The particles may also have a narrow size distribution such that 90% or more of the particles by volume (D90) have a diameter within the ranges noted above. A variety of different materials may be employed to form such particles, such as fatty acids or salts thereof (e.g., stearic acid, citric acid, myristic acid, palmitic acid, linoleic acid, etc., as well as salts thereof), cellulosic compounds (e.g., hydroxymethylcellulose, carboxymethylcellulose, ethylcellulose, methylcellulose, etc.), biocompatible salts (e.g., sodium chloride, calcium chloride, sodium phosphate, etc.), hydroxy-functional compounds, and so forth. In particularly suitable embodiments, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof.

One or more nonionic, anionic, and/or amphoteric surfactants may also be employed such as described above to help create a uniform dispersion. When employed, such surfactant(s) typically constitute from about 0.05 wt. % to about 8 wt. %, and in some embodiments, from about 0.1 wt. % to about 6 wt. %, and in some embodiments, from about 0.5 wt. % to about 3 wt. % of the membrane layer.

The membrane layer(s) may be formed using the same or a different technique than used to form the core, such as by hot-melt extrusion, injection molding, solvent casting, dip coating, spray coating, microextrusion, coacervation, etc. In one embodiment, a hot-melt extrusion technique may be employed. The core and membrane layer(s) may also be formed separately or simultaneously. In one embodiment, for instance, the core and membrane layer(s) are separately formed and then combined together using a known bonding technique, such as by stamping, hot sealing, adhesive bonding, etc. Compression molding (e.g., vacuum compression molding) may also be employed to form the implantable device. As described above, the drug release and membrane layer(s) may be each individually formed by heating and compressing the respective polymer compression into the desired shape while under vacuum. Once formed, the drug release and membrane layer(s) may be stacked together to form a multi-layer precursor and thereafter compression molded in the manner as described above to form the resulting implantable device.

### IV. Use of Device

Through selective control over the particular nature of the device and the manner in which it is formed, the resulting device can be effective for sustained release over a nucleic acid over a prolonged period of time. For example, the implantable medical device can release the nucleic acid for a time period of about 5 days or more, in some embodiments about 10 days or more, in some embodiments from about 20 days to about 60 days, and in some embodiments, from about 25 days to about 50 days (e.g., about 30 days). Further, the nucleic acid can be released in a controlled manner (e.g., zero order or near zero order) over the course of the release time period. After a time period of about 7 days, for example, the cumulative release ratio of the implantable medical device may be from about 5% to about 60%, such as from about 10% to about 40%. In another example, after a time period of 15 days, the cumulative release ratio of the implantable medical device may be from about 20% to about 70%, in some embodiments from about 30% to about 65%, and in some embodiments, from about 40% to about 60%. Likewise, after a time period of 30 days, the cumulative release ratio of the implantable medical device may still be from about 40% to about 85%, in some embodiments from about 50% to about 80%, and in some embodiments, from about 60% to about 80%. The “cumulative release ratio” may be determined by dividing the amount of the nucleic acid released at a particulate time interval by the total amount of nucleic acid initially present, and then multiplying this number by 100.

In embodiments, the cumulative release ratio of the implantable device can vary depending on the materials of the core and/or membrane layers, including therapeutic agent loading and membrane configuration. For instance, in embodiments, at a time period of about 10 days the cumulative release ratio is from about 1% to less than about 20%. In other embodiments, at a time period of about 10 days the cumulative release ratio is from about 60% to about 80%. In still other embodiments, at a time period of about 40 days the cumulative release ratio is from about 25% to 35%. In other embodiments, at a time period of about 40 days the cumulative release ratio is about 40% to about 60%. In another embodiment, at a time period of about 40 days the cumulative release ratio is from about 70% to about 80%. In other embodiments, at a time period of about 60 days, the cumulative release ratio is about 30% to about 50%. In other embodiments, at a time period of about 60 days, the cumulative release ratio is about 50% to 70%. Still in another embodiment, at a time period of about 60 days the cumulative release ratio is about 75% to about 90%.

Of course, the actual dosage level of the nucleic acid delivered will vary depending on the particular nucleic acid employed and the time period for which it is intended to be released. The dosage level is generally high enough to provide a therapeutically effective amount of the nucleic acid to render a desired therapeutic outcome, i.e., a level or amount effective to reduce or alleviate symptoms of the condition for which it is administered. The exact amount necessary will vary, depending on the subject being treated, the age and general condition of the subject to which the nucleic acid is to be delivered, the capacity of the subject's immune system, the degree of effect desired, the severity of the condition being treated, the particular nucleic acid selected and mode of administration of the composition, among other factors. An appropriate effective amount can be readily determined by one of skill in the art. For example, an effective amount will typically range from about 5 μg to about 200 mg, in some embodiments from about 5 μg to about 100 mg per day, and in some embodiments, from about 10 μg to about 1 mg of the nucleic acid delivered per day.

The device may be implanted subcutaneously, orally, mucosally, etc., using standard techniques. The delivery route may be intrapulmonary, gastroenteral, subcutaneous, intramuscular, into the central nervous system (e.g., intrathecal), intraperitoneum, intraorgan, etc. In one embodiment, the implantable device may be particularly suitable for delivering a nucleic acid for cancer treatment. In such embodiments, the device may be placed in a tissue site of a patient in, on, adjacent to, or near a tumor, such as a tumor of the pancreas, biliary system, gallbladder, liver, small bowel, colon, brain, lung, eye, etc. The device may also be employed together with current systemic chemotherapy, external radiation, and/or surgery. The device may also be delivered intrathecally. In such embodiments, the device may be implanted into the spinal canal or directly into the intrathecal space (subarachnoid space), which is the space that holds the cerebrospinal fluid. For example, intrathecal administration may be accomplished by implanting the device into an Ommaya reservoir (a dome-shaped container that is placed under the scalp during surgery; it holds the drugs as they flow through a small tube into the brain) or directly into the cerebrospinal fluid in the lower part of the spinal column.

The device can also be inserted into the eye of a patient. For instance, the device can be intraocularly inserted into the patient's eye. To avoid having to pass through the blood-aqueous barrier, the implantable device may be directly inserted into the posterior segment, such as into the vitreous humor (“intravitreally”). Intravitreal injection techniques are well known in the art any may include, for instance, the use of a hollow needle through which the implant is passed. The needle may have a small diameter size (e.g., 18 to 30 gauge needle) so that the incision is self-sealing, and the implantation occurs in a closed chamber. A self-sealing incision may also be formed using a conventional “tunneling” procedure in which a spatula-shaped scalpel is used to create a generally inverted V-shaped incision through the cornea. In a preferred mode, the instrument used to form the incision through the cornea remains in place (that is, extends through the corneal incision) during the procedure and is not removed until after implantation. Such incision-forming instrument either may be used to place the ocular implant or may cooperate with a delivery instrument to allow implantation through the same incision without withdrawing the incision-forming instrument. Of course, in other modes, various surgical instruments may be passed through one or more corneal incisions multiple times.

The implantable device can be placed in the desired location within the eye according to surgical procedures, such as those known in the art. For example, the implantable device can be inserted through a sclerotomy into the suprachoroid. In such a procedure, the sclera is cut to expose the suprachoroid. The implantable device can then be positioned and inserted on either side of the incision. Alternatively, a partial-thickness scleral trap-door can be fashioned over the suprachoroid or an avascular region of the eye. The implantable device can then be inserted, and the scleral flap sewn back into place to secure the implant. Alternatively, the implantable device may be inserted so as to directly communicate with the vitreal chamber. To achieve this, a partial thickness scleral trap door flap is cut over an avascular region, such as the pars plana, to remove the eye coat. A hole (or holes) is made through the floor of the scleral bed to communicate with the base of the vitreous body through the pars plana. The implantable device is positioned over the hole within the scleral bed and the flap of the trap door is sewn back into place. Such placement of the implantable device will allow for the ready diffusion of the drug into the vitreous and into the intraocular structure.

Once implanted, the implantable device can be used to treat and/or prohibit a variety of different conditions. Conditions that can be treated and/or prohibited include cancers (e.g., breast cancer, thyroid cancer, lung cancer, prostate cancer, cervical cancer, ovarian cancer, skin cancer, bowel cancer, colorectal cancer, uterine cancer, blood cancers, bone cancers), neurological diseases (e.g., neurodegenerative disease, such as spinal muscular atrophy, amyotrophic lateral sclerosis, multiple sclerosis), epilepsy, stroke, brain aneurysm, Parkinson's disease, bipolar disorder, schizophrenia, dementia, Alzheimer's disease, depression, pain management, viral infections, bacterial infections, allergies, asthma, lung disease, cystic fibrosis, COPD, emphysema, tuberculosis, gastrointestinal diseases, autoimmune diseases (e.g., Graves' disease, Lichen planus, psoriasis, vitiligo, Addison's disease, celiac disease, Crohn's disease, ulcerative colitis, Sjogren's syndrome, aplastic anemia, ankylosing spondylitis, Lyme disease, sarcoidosis, fibromyalgia, myasthenia gravis, multiple sclerosis, optic neuritis, restless leg syndrome, Meniere's disease, Kawasaki disease), heart diseases (e.g., atherosclerosis, pericarditis, rheumatic heart disease), liver diseases (e.g., hepatitis, non-alcoholic fatty liver disease, cirrhosis), arthritis (e.g., osteoarthritis and rheumatoid arthritis), osteomyelitis, osteoporosis, cervical spondylosis, skin diseases (e.g., atopic dermatitis, dermatitis, seborrheic dermatitis, eczema, folliculitis), scleroderma, metabolic diseases (e.g., Type-1 Diabetes and Type-2 Diabetes), Lupus, kidney diseases (e.g., kidney failure, nephrotic syndrome, glomerulonephritis), eye diseases (e.g., cytomegalovirus retinitis, glaucoma), hyperthyroidism, hypothyroidism, insomnia, paralysis, sciatica, Bell's Palsy, hypertension, and thalassemia.

If desired, the device may be sealed within a package (e.g., sterile blister package) prior to use. The materials and manner in which the package is sealed may vary as is known in the art. In one embodiment, for instance, the package may contain a substrate that includes any number of layers desired to achieve the desired level of protective properties, such as 1 or more, in some embodiments from 1 to 4 layers, and in some embodiments, from 1 to 3 layers. Typically, the substrate contains a polymer film, such as those formed from a polyolefin (e.g., ethylene copolymers, propylene copolymers, propylene homopolymers, etc.), polyester (e.g., polyethylene terephthalate, polyethylene naphthalate, polybutylene terephthalate, etc.), vinyl chloride polymer, vinyl chloridine polymer, ionomer, etc., as well as combinations thereof. One or multiple panels of the film may be sealed together (e.g., heat sealed), such as at the peripheral edges, to form a cavity within which the device may be stored. For example, a single film may be folded at one or more points and sealed along its periphery to define the cavity within with the device is located. To use the device, the package may be opened, such as by breaking the seal, and the device may then be removed and implanted into a patient.

### EXAMPLES

### Test Methods

Drug Release: The release of an oligonucleotide from a polymeric implant may be determined using an in vitro method. More particularly, implantable device samples may be placed in 5 milliliters of an aqueous PBS buffer solution or a Tris EDTA(TE) buffer solution. The solutions are enclosed in centrifuge tubes. The tubes are then placed into a temperature-controlled incubator and continuously shaken at 100 rpm. A temperature of 37° C. is maintained through the release experiments to mimic in vivo conditions. Samples are taken in regular time intervals by completely exchanging the buffer solution. The concentration of an oligonucleotide in solution may be determined via an HPLC method which is described below. From these data, the amount of the oligonucleotide released per sampling interval (milligram per day) may be calculated and plotted over time (days). Further, the cumulative release ratio of the oligonucleotide may be calculated as a percentage by dividing the amount of the oligonucleotide released at each sampling interval by the total amount of oligonucleotide initially present, and then multiplying this number by 100. This percentage is then plotted over time (days).

Antisense HPLC Method: An Agilent 1260 series HPLC system equipped with a Gen-Pak Anion-Exchange Column (2.5 μm, 4.6 mm×100 mm) was used. The gradient HPLC method was used to analyze the samples at 25° C. With a flow rate of 0.5 ml/min, the mobile phase was made up of solvent A (100 mM TRIS base in water, pH 8, 15% acetonitrile) and solvent B (100 mM TRIS base, 2M Sodium bromide in water, pH 8, 15% acetonitrile). The initial mobile phase condition was 100% solvent A and 0% solvent B, which was changed linearly (0-25 min) to 10% solvent A and 90% solvent B, and then returned to the initial conditions within 5 minutes (25-30 min). The entire run took 30 minutes. A 10 μL sample was injected and absorbance at 260 nm was used for detection.

### Example 1

A model 20 mer antisense oligonucleotide (ASO) was purchased from Oligo Factory. The ASO was crushed into smaller particle sizes so that it could be blended with EVA powder. A phosphorothioate deoxyribose backbone was used for the bases at the center of the chain of the ASO. Several units at each end of the chain had a phosphorothioate ribose backbone that was 2′-O-methylated. The sequence is shown below.

5′ mA-mU-mC-mA-mG-dT-dC-dT-dT-dT-dT-dC-dC-dT-dC-mU-mA-mC-mG-mA 3′

Where: m=2′-O-methoxyethyl ribonucleoside; and d=Deoxynucleoside. Each of m or d are conjugated to at least one nucleotide base including adenine (A), cytosine (C), guanine (G), thymine (T), or uracil (U).

A rod-shaped monolithic implant containing ASO was produced via extrusion. The device contained 50 wt. % Ateva® 4030AC and 50 wt. % ASO. The device was formed by melt extruding the components using a 11 mm twin-screw extruder. Extrusion was accomplished using a screw speed of 50 rpm with barrel temperatures set to achieve a nominal melt temperature of 63° C. Example Processing Conditions are shown below.

Process Conditions
Heat Profile (C. °)

| | |
|---|---|
| Zone 2 | 50 |
| Zone 3 | 65 |
| Zone 4 | 65 |
| Zone 5 | 65 |
| Zone 6 | 65 |
| Zone 7 | 65 |
| Zone 8 | 65 |
| Die | 65 |

Readings

| | |
|---|---|
| Pressure (Bar) | 32 |
| Torque (Nm) | 2.5 |
| Speed (RPM) | 50 |
| Melt | 63 |
| Air (PSI) | 0 |

Feeder Speeds

| | |
|---|---|
| Feeder 1 | N/A |
| Feeder 2 | N/A |

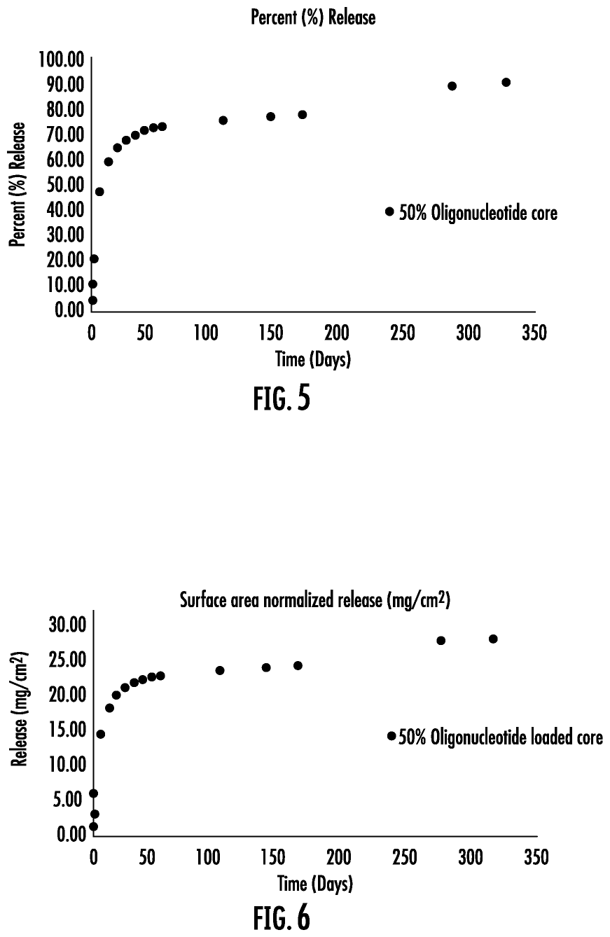

The extruded rods had a diameter of approximately 2 mm and were cut to a length of 1 cm for elution testing. Six samples were tested in parallel to facilitate the characterization of experimental error. The release of ASO from the rods was measured in PBS buffer in a shaking incubator maintained at 37° C. At regular intervals, the buffer was exchanged with fresh buffer, and the removed buffer characterized using the previously described HPLC method to measure the concentration of ASO released. Individual release curves for each of the six samples were determined, and then the average and standard deviation cumulative release of the six samples was calculated at each time point. The resulting cumulative release rate (%) over 7 days is shown in FIG. 5 (results shown in Table 1 below) and the surface area normalized release rate is shown in FIG. 6 (results shown in Table 2 below).

TABLE 1
Percent drug release from 50% loaded core (FIG. 5)

| Time | Average (%) |
|---|---|
| (Days) | Release |
| 1 | 4.10 |
| 2 | 9.86 |
| 3 | 19.51 |
| 7 | 46.13 |
| 14 | 57.90 |
| 21 | 63.20 |
| 28 | 66.41 |
| 35 | 68.55 |
| 42 | 70.06 |
| 49 | 71.22 |
| 56 | 72.10 |
| 106 | 74.82 |
| 143 | 76.03 |
| 169 | 76.76 |
| 287 | 87.86 |
| 330 | 88.92 |

TABLE 2
Surface area normalized drug release
from 50% loaded core (FIG. 6)

| Time | Average (%) |
|---|---|
| (Days) | Release |
| 1 | 1.26 |
| 2 | 3.04 |
| 3 | 6.02 |
| 7 | 14.36 |
| 14 | 18.05 |
| 21 | 19.70 |
| 28 | 20.70 |
| 35 | 21.38 |
| 42 | 21.85 |
| 49 | 22.21 |
| 56 | 22.49 |
| 106 | 23.34 |
| 143 | 23.73 |
| 169 | 23.96 |
| 287 | 27.43 |
| 330 | 27.76 |

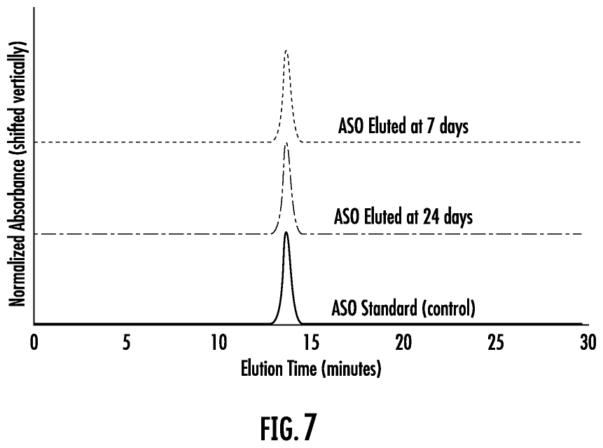

HPLC chromatograms, as shown in FIG. 7, taken from the elution media collected at each time point, were compared with a standard control solution of unextruded ASO. This comparison enables the evaluation of the stability of the ASO through the extrusion process and during the in vitro release testing. FIG. 7 compares the ASO control solution with the elution media collected after 24 hours of release and after 7 days of release. The normalized HPLC curves are essentially the same, which suggests that the ASO has not been chemically degraded or altered during extrusion or the in vitro release study.

### Examples 2-9

Rod-shaped implants containing the ASO as described in Example 1 were produced via extrusion. Examples 2-4 were monolithic implants containing no membrane layer. Drug loading and composition of Examples 2-4 are shown in Table 3 below. Example 2 contained 50 wt. % ASO and 50 wt. % Ateva® 4030AC (having a vinyl acetate content of 40 wt. %). Example 3 contained 60 wt. % ASO and 40 wt. % Ateva® 4030AC. Example 4 contained 70 wt. % ASO and 30 wt. % Ateva® 4030AC. To prepare Examples 5-9, extruded cores containing 60 wt. % or 70 wt. % ASO were remolded via vacuum compression molding to yield rods having a length of 10 mm and a diameter of 2 mm. Membrane compositions were then applied to the cores via vacuum compression molding. The final diameters of Examples 5-9 ranged from about 2.3 mm to about 3 mm. Drug loading and compositions of Examples 5-9 are shown in Table 4 below. Example 5 includes a core having 60 wt. % ASO and 40 wt. % Ateva® 4030AC and a membrane (thickness 0.25 mm) containing 35 wt. % mannitol (25 μm particle size) and 65 wt. % Ateva® 4030AC. Example 6 includes a core having 60 wt. % ASO and 40 wt. % Ateva® 4030AC and a membrane (thickness 0.5 mm) containing 35 wt. % mannitol (25 μm particle size). Example 7 includes a core having 70 wt. % ASO and 30 wt. % Ateva® 4030AC and a membrane (thickness 0.25 mm) containing 35 wt. % mannitol (25 μm particle size) and 65 wt. % Ateva® 4030AC. Example 8 includes a core having 70 wt. % ASO and 30 wt. % Ateva® 4030AC and a membrane (thickness 0.5 mm) containing 35 wt. % mannitol (25 μm particle size) and 65 wt. % Ateva® 4030AC. Example 9 includes a core having 70 wt. % ASO and 30 wt. % Ateva® 4030AC and a membrane (thickness 0.25 mm) containing 20 wt. % Solplus and 80 wt. % Ateva® 4030AC.

TABLE 3

| | Monolithic implants (no membrane) | | |
|---|---|---|---|
| | Example 2 | Example 3 | Example 4 |
| Antisense | 50 wt. % | 60 wt. % | 70 wt. % |
| Oligonucleotide | | | |
| Ateva ® 4030AC | 50 wt. % | 40 wt. % | 30 wt. % |

TABLE 4

| Implants with Membranes | | | | |
|---|---|---|---|---|
| Example 5 | Example 6 | Example 7 | Example 8 | Example 9 |

Core Composition

| | | | | | | | | | | |
|---|---|---|---|---|---|---|---|---|---|---|
| Antisense | 60 | wt.% | 60 | wt.% | 70 | wt.% | 70 | wt.% | 70 | wt.% |
| Oligonucleotide | | | | | | | | | | |
| Ateva ® 4030AC | 40 | wt.% | 40 | wt.% | 30 | wt.% | 30 | wt.% | 30 | wt.% |

Membrane Composition

| Membrane | 35 wt. % | | 35 wt. % | | 35 wt. % | | 35 wt. % | | 20 wt. % | |
|---|---|---|---|---|---|---|---|---|---|---|
| composition | mannitol + | | mannitol + | | mannitol + | | mannitol + | | Soluplus + | |
| | 65 wt. % | | 65 wt. % | | 65 wt. % | | 65 wt. % | | 80 wt. % | |
| | Ateva ® | | Ateva ® | | Ateva ® | | Ateva ® | | Ateva ® | |
| | 4030AC | | 4030AC | | 4030AC | | 4030AC | | 4030AC | |
| Membrane | 0.25 | mm | 0.5 | mm | 0.25 | mm | 0.5 | mm | 0.25 | mm |
| thickness | | | | | | | | | | |

| | | | | | | | | | |
|---|---|---|---|---|---|---|---|---|---|
| Mannitol | 25 | μm | 25 | μm | 25 | μm | 25 | μm | NA |

Average
particle size

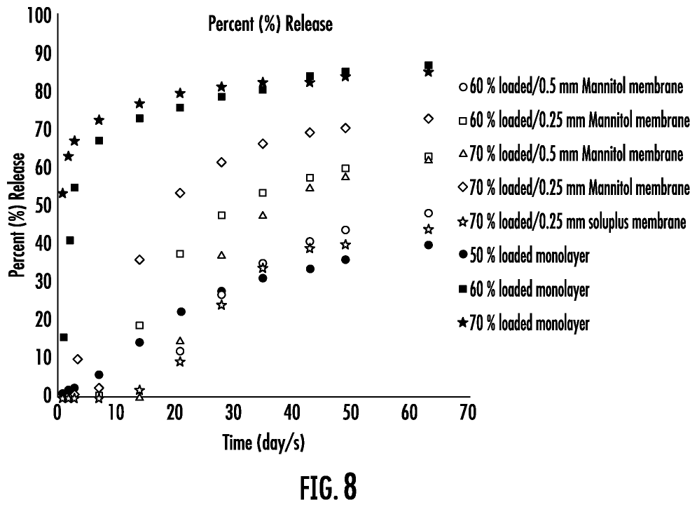

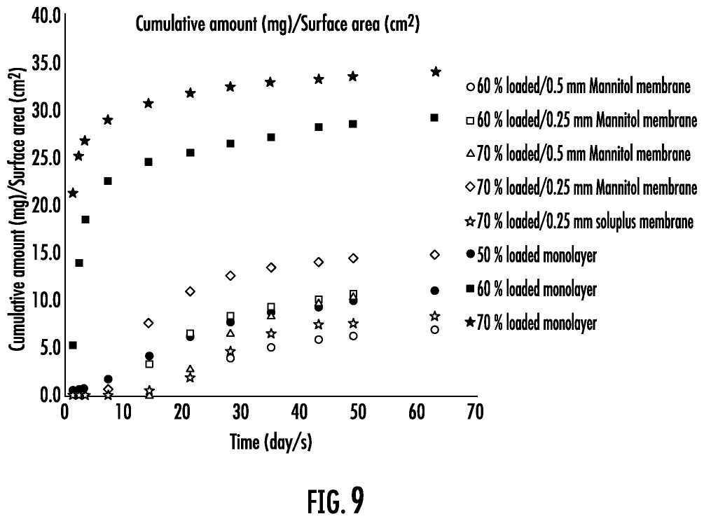

The implants were then used for elution testing according to the test methods described above. For Example 2, the release of ASO from the rods was measured in PBS buffer in a shaking incubator maintained at 37° C. At regular intervals, the buffer was exchanged with fresh buffer, and the removed buffer characterized using the previously described HPLC method to measure the concentration of ASO released. The resulting cumulative release rate (%) is shown in FIG. 8 (results shown in Tables 5-6 below). For Examples 3-9, the release of ASO from the rods was measured in Tris EDTA (TE) buffer in a shaking incubator maintained at 37° C. At regular intervals, the buffer was exchanged with fresh buffer, and the removed buffer characterized using the previously described HPLC method to measure the concentration of ASO released. The resulting cumulative release rates for Examples 2-9 (%) are shown in FIG. 8. Surface area normalized release rates for Examples 2-9 are shown in FIG. 9 (results shown in Table 7-8 below).

TABLE 5
Total percent release (FIG. 8)

| Time | | | |
|---|---|---|---|
| (Days) | Ex. 2 | Ex. 3 | Ex. 4 |
| 1 | 1.104499 | 15.66016 | 53.24464 |
| 2 | 1.77885 | 41.19219 | 63.14128 |
| 3 | 2.461801 | 55.07306 | 66.92265 |
| 7 | 6.021707 | 67.3447 | 72.61614 |
| 14 | 14.46126 | 73.15397 | 77.04636 |
| 21 | 22.28964 | 76.13146 | 79.59304 |
| 28 | 27.79712 | 78.7055 | 81.27711 |
| 35 | 31.65955 | 80.78686 | 82.53527 |
| 43 | 33.73152 | 83.78204 | 83.5463 |
| 49 | 36.00314 | 84.96831 | 84.17625 |
| 63 | 39.74417 | 87.02541 | 85.01416 |

TABLE 6
Total percent release (FIG. 8)

| Time | | | | | |
|---|---|---|---|---|---|
| (days) | Ex. 5 | Ex. 6 | Ex. 7 | Ex. 8 | Ex. 9 |
| 1 | 0 | 0.013019 | 0 | 0.043471 | 0 |
| 2 | 0 | 0.01608 | 0 | 0.049082 | 0 |
| 3 | 0 | 0.031121 | 0 | 0.096652 | 0 |
| 7 | 0.00273698 | 0.409087 | 0.0009488 | 2.46943 | 0.002812 |
| 14 | 0.11350666 | 19.11062 | 0.0092321 | 36.01171 | 1.819691 |
| 21 | 12.2647037 | 37.52576 | 14.735464 | 53.23943 | 9.420121 |
| 28 | 27.1675604 | 47.82479 | 36.935121 | 61.68717 | 24.12848 |
| 35 | 35.2454927 | 53.48573 | 47.819435 | 66.33159 | 34.5745 |
| 43 | 40.9366568 | 57.57565 | 54.634743 | 69.30904 | 39.06184 |
| 49 | 43.993607 | 59.84668 | 57.877071 | 70.70941 | 39.94103 |
| 63 | 48.3350416 | 62.9315 | 62.505266 | 72.75485 | 44.08185 |

TABLE 7
Cumulative release surface area normalized (mg/cm²) (FIG. 9)

| Time | | | |
|---|---|---|---|
| (Days) | Ex. 2 | Ex. 3 | Ex. 4 |
| 1 | 0.3048 | 5.2627 | 21.2519 |
| 2 | 0.4906 | 13.8408 | 25.2020 |
| 3 | 0.6802 | 18.5013 | 26.7109 |
| 7 | 1.6587 | 22.6233 | 28.9834 |
| 14 | 3.9751 | 24.5759 | 30.7514 |
| 21 | 6.1359 | 25.5762 | 31.7675 |
| 28 | 7.6583 | 26.4411 | 32.4392 |
| 35 | 8.7265 | 27.1398 | 32.9412 |
| 43 | 9.3007 | 28.1453 | 33.3445 |
| 49 | 9.9308 | 28.5437 | 33.5958 |
| 63 | 10.9701 | 29.2341 | 33.9300 |

TABLE 8
Cumulative release surface area normalized (mg/cm²) (FIG. 9)

| Time | | | | | |
|---|---|---|---|---|---|
| (day/s) | Ex. 5 | Ex. 6 | Ex. 7 | Ex. 8 | Ex. 9 |
| 1 | 0.0000 | 0.0022 | 0.0000 | 0.0086 | 0.0000 |
| 2 | 0.0000 | 0.0027 | 0.0000 | 0.0098 | 0.0000 |
| 3 | 0.0000 | 0.0052 | 0.0000 | 0.0192 | 0.0000 |
| 7 | 0.0004 | 0.0683 | 0.0002 | 0.5035 | 0.0005 |
| 14 | 0.0165 | 3.2724 | 0.0016 | 7.4589 | 0.3431 |
| 21 | 1.7474 | 6.4643 | 2.6327 | 10.8756 | 1.7677 |
| 28 | 3.8456 | 8.2634 | 6.5168 | 12.5493 | 4.5368 |
| 35 | 4.9868 | 9.2484 | 8.4144 | 13.4671 | 6.4998 |
| 43 | 5.7889 | 9.9611 | 9.5994 | 14.0565 | 7.3473 |
| 49 | 6.2186 | 10.3546 | 10.1619 | 14.3340 | 7.5186 |
| 63 | 6.8275 | 10.8893 | 10.9622 | 14.7387 | 8.2903 |

These and other modifications and variations of the present disclosure may be practiced by those of ordinary skill in the art, without departing from the spirit and scope of the present disclosure. In addition, it should be understood that aspects of the various embodiments may be interchanged both in whole or in part. Furthermore, those of ordinary skill in the art will appreciate that the foregoing description is by way of example only and is not intended to limit the disclosure so further described in such appended claims.

What is claimed is:

1. An implantable medical device comprising a core comprising an antisense oligonucleotide (ASO) dispersed within a core polymer matrix comprising an ethylene vinyl acetate copolymer, the ASO comprising one or more nucleosides attached via internucleoside linkages, wherein at least 10% of the internucleoside linkages comprise chemically modified internucleoside linkages, wherein from about 5% to about 60% of the ASO is released from the implantable medical device after a time period of about seven days.

2. The implantable medical device of claim 1, wherein a weight ratio of the polymer matrix to the ASO is from about 1 to about 10.

3. The implantable medical device of claim 1, wherein the ethylene vinyl acetate copolymer has a melting temperature of from about 20° C. to about 100° C. as determined in accordance with ASTM D3418-15.

4. The implantable medical device of claim 1, wherein the ethylene vinyl acetate polymer has a melt flow index of from about 0.2 to about 100 grams per 10 minutes as determined in accordance with ASTM D1238-20 at a temperature of 190° C. and a load of 2.16 kilograms.

5. The implantable medical device of claim 1, wherein the vinyl acetate content of the copolymer is from about 10 wt. % to about 60 wt. %.

6. The implantable medical device of claim 1, wherein the ASO has a length of from about 8 nucleosides subunits to about 50 nucleosides subunits.

7. The implantable medical device of claim 1, wherein at least 10% to 90% of the internucleoside linkages in the backbone of the ASO comprise chemically modified internucleoside linkages.

8. The implantable medical device of claim 1, wherein the chemically modified internucleoside linkages comprise phosphorothioate linkages.

9. The implantable medical device of claim 1, wherein the chemically modified internucleoside linkages comprise morpholino subunits.

10. The implantable medical device of claim 1, wherein the ASO comprises a gap region having at least 8 to about 10 contiguous 2′-deoxyribonucleosides and a first wing region and a second wing region flanking the gap region, wherein each of the first and second wing regions independently have 1 to 8 2′-O-(2-methoxyethyl) ribonucleotides.

11. The implantable medical device of claim 10, wherein internucleoside linkages in the first wing region and the second wing region comprise phosphorothioate linkages.

12. The implantable medical device of claim 10, wherein at least 75% of the internucleoside linkages in the gap region comprise phosphorothioate linkages.

13. The implantable medical device of claim 1, wherein the ASO comprises a 5-10-5 gapmer.

14. The implantable medical device of claim 1, wherein the ASO constitutes from about 50 wt. % to about 70 wt. % of the core and the polymer matrix constitutes from about 50 wt. % to about 30 wt. % of the core.

15. The implantable medical device of claim 1, further comprising a membrane layer positioned adjacent to an outer surface of the core, wherein the membrane layer comprises a membrane polymer matrix comprising a hydrophobic polymer and a hydrophilic compound.

16. The implantable medical device of claim 1, wherein at a time period of about 10 days the cumulative release ratio of the ASO is between about 60% to about 80%.

17. A method of manufacturing an implantable medical device, the method comprising:
melt-blending a first polymer matrix containing an ethylene vinyl acetate copolymer and an ASO in an extruder barrel at a first temperature, the ethylene vinyl acetate copolymer having a melt flow index of from about 1 to about 400 grams per 10 minutes as determined in accordance with ASTM D1238-20 at a temperature of 190° C. and a load of 2.16 kilograms;
mixing the first polymer matrix and ASO in the extruder barrel at a second temperature to form a mixture of core polymer matrix and therapeutic agent;
extruding the mixture of core polymer matrix and therapeutic agent from the extruder barrel forming a core of the implantable device;
cooling the core; and
cutting the core to form the implantable medical device.

18. The method of claim 17, wherein the first temperature is from about 30° C. to about 60° C., preferably about 50° C.

19. The method of claim 17, wherein the second temperature is from about 60° C. to about 70° C., preferably about 65° C.

20. The method of claim 17, wherein the extruder barrel includes a rotatable screw having a length and a diameter, wherein a ratio of the length to the diameter is from about 10 to about 50.
